(19) United States
(12) Patent Application Publication
(10) Pub. No.: US 2002/0182245 A1
(43) Pub. Date: Dec. 5, 2002

(54) Hydrophilic polyurethane as a matrix for the delivery of skin care ingredients

(76) Inventor: Timothy Thomson, West Newbury, MA (US)

Correspondence Address: William B. Ritchie, Ritchie Law Offices, 43 Jackson Street, Concord, NH, 03301, US

(21) Appl. No.: 10/114,336

(22) Filed: Apr. 2, 2002

Related U.S. Application Data
(60) Provisional application No. 60/281,099, filed on Apr. 3, 2001.

(51) Int. Cl. A61K 9/70

(52) U.S. Cl. 424/443

(57) ABSTRACT

A cosmetic delivery system that uses the chemically and physically stable nature of a hydrophilic polyurethane sponge as the medium, as opposed to creams or lotions that require surfactants, emulsifiers, stabilizers and thickeners.

### FIELD OF THE INVENTION

The present invention relates to a device for the delivery of skin care active ingredients by the use of hydrophilic polyurethane sponges and without the use of emulsifying agents and stabilizers.

### BACKGROUND OF THE INVENTION

Creams and Lotions are emulsions, either oil in water or water in oil. For cosmetic applications, oil in water emulsion is typical. For these systems, water is said to be the continuous phase and the oil phase is distributed in the water as small particles of less than 0.5 μM. The technology to produce stable emulsions of what would normally be incompatible phases is by the addition of another chemical that is water soluble on one end and oil soluble on the other. These chemicals are called surfactants or emulsifiers. Thus, if water, oil and the proper surfactant are mixed together, the blend will organize into a structure, all stabilized by the surfactant. In most cases, several surfactants are used.

Still further, other components are added to increase the viscosity of the cream or lotion. These further stabilize the system and permit claims of long shelf life. In many cases, the thickeners are surfactants in themselves. These can contribute to or degrade the effectiveness of the surfactants. Thus still more components are added to further stabilize the lotion.

A cream or a lotion, therefore, is typically composed of water, an emulsifier, a thickener and an oil-phase. Many times fragrances are added. These components are processed in such as way as to produce a stable, aesthetically pleasing product. It is not uncommon for a commercial cream or lotion to contain as many as 20 components, most of which are there to produce and stabilize the emulsion of an active ingredient and water.

U.S. Pat. No. 2,204,202 discloses a latex foam pad within which is placed a product that is to be placed on a user. The relatively high density and surface skin of this device inhibits the delivery of the product to the user.

U.S. Pat. No. 5,762,946 discloses a multi-layered device intended to be a cosmetic delivery pad. A foam layer is said to contain an active ingredient. Examples of ingredients that might be included are cosmetic, dermatological or pharmaceutical products for topical application. Specific mention is made of soaps, shampoos, make-up removers, sun products, care creams, plant and animal extracts, vitamins, proteins, enzymes, mineral salts, trace elements, etc. The patent teaches the use of polyurethane as a suitable foam, but it is classed with many other foams including polyesters, polyethers, polyethylene, polystyrene and polyvinyl chloride.

In as much as all of the other foams are hydrophobic, the mechanism by which the device disclosed in the '946 patent functions is as a scaffold on which the active ingredients are coated. The '946 does not teach of the use of hydrophilic polyurethanes which is a class of polyurethanes that comprises less than 0.1% of all polyurethanes. Nor does the '946 patent recognize the ability of this class of polyurethanes is due to their hydrophilic nature, that is, to contain the active ingredients in the matrix of the foam itself. Thus, while '946 patent teaches the use of polyurethanes as a scaffold, it does not disclose or suggest the use of hydrophilic polyurethane as a matrix within which an active ingredient is dispersed.

U.S. Pat. No. 4,806,572 discloses the use of hydrophilic polyurethane sponge for the delivery of make up remover formulations. The formulations discussed are complex and do not produce formulations that minimize the use of non-active ingredients, particularly surfactants, emulsifiers and thickeners. Further, the method of preparation is by the emulsification of an aqueous phase containing the make up remover components and a hydrophilic polyurethane prepolymer. The inventor has found that this limits the types of components that can be incorporated into the foam. If, for instance, the active ingredient has alcohol or carboxylic acid ligands or end groups, the isocyanate reacts with them. This immobilizes that component, thus making it unavailable for delivery. Secondly, the reaction typically will not abstract a carbon dioxide molecule and, further, the active ingredient serves as a chain-terminating molecule. In this case the density and quality is affected.

An application apparatus that is able to apply active ingredients in a typical cream or lotion to the skin without the use of the surfactants, emulsifiers, and the like, which are necessary to make a stable emulsion using prior art techniques, has both marketing and toxicological advantages.

### SUMMARY OF THE INVENTION

It is an aspect of the present invention to provide a foam pad that contains an active ingredient for the purpose of distributing a topical treatment product.

It is another aspect of the present invention to provide a foam pad that can be used for the application of make up.

Still another aspect of the present invention is to provide a foam that can be used to cleanse the skin of a user.

Another aspect of the present invention to provide a foam pad that can apply sun screen to a user.

It is an aspect of the present invention to provide a foam pad that has a moisturizer that can be applied to the skin.

Another aspect of the present invention is to provide a foam pad that has incorporated one or more ingredients selected from the group consisting of plant extracts, vitamins, proteins, amino acids, fatty acids, and other chemical products.

Still a further aspect of the invention is to provide a foam pad that can be utilized to provide a dermatological agent to an animal to treat a medical condition.

Finally, it is an aspect of the invention to process for fabricating a foam pad that washes the foam to remove residual surfactants and then to imbibe an active ingredient into the foam.

These and other aspects of the invention will become apparent in light of the detailed description of the invention which follows.

### DETAILED DESCRIPTION OF THE PREFERRED EMBODIMENTS

Hydrophilic polyurethane sponges are made by what is commonly referred to as the “Prepolymer Process”. A prepolymer in this connection is an isocyanate-capped polyol. Conventional (hydrophobic) polyurethanes can be made by this process. In that case, however, a hydrophobic polyol is used. In the case of a hydrophilic polyurethane, however, a hydrophilic polyol, usually a polyethylene glycol, is used. In a typical process, an aqueous phase and the prepolyiner are emulsified. A chemical reaction takes place between the water in the aqueous and the isocyanates to liberate carbon dioxide gas and, simultaneously, polymerize the mass. The result is a chemically and physically stable, low-density sponge.

The inventor has discovered that key to the solution to the problem is to separate the foam making process from the process by which an active ingredient is added. This enables the invention to use the hydrophilic polyurethane foam structure as the delivery matrix. The use of the teachings discovered by the inventor enables the inclusion of a wider variety of active ingredients in a foam of a desired texture and cell size.

Each active ingredient that is to be used with the invention has an individual and unique delivery characteristic dependent on its solubility of the component. The inventor has discovered that by using a hydrophilic polyurethane foam, the active ingredients are encapsulated in the matrix of the foam as opposed to coating the surface of the matrix. This makes the delivery rate a function of the diffusion out of the matrix the rate controlling step as opposed to simple solubility. The diffusion is more typically a characteristic of the matrix. This has the effect of moderating the release characteristics.

The quality of the foam is in part governed by the quality of the emulsion that is created. To aid in the emulsion process, a number of surfactants are typically used. The following table from a Hypol brochure illustrates the technique.

| Surfactant | Source | Result |
|---|---|---|
| Pluronic | BASF | Fine, open celled |
| L62 | | |
| Pluronic | BASF | Larger open cells |
| F88 | | |
| Brij72 | ICI America | Fine cells supersoft |
| L-520 | Union Carbide | Fine cells non-wicking |
| DC190 | Dow-Corning | Hydrophobic open cell |

While other factors including temperature and mixer speed also contribute to the foam structure, it is common for the manufacturer to use the surfactant to control foam quality. It is clear that adding another component to the formulation, particularly a surface active ingredient, will affect the foam quality.

Once made, however, the foam is known to be both chemically and physically stable.

After the foam is made, it is washed to remove residual surfactants and then to imbibe an active ingredient into the foam. Thus we are able to control the quality of the foam while still being able to use it as a stabilizing medium for the active ingredient without the use of surfactants, emulsifiers, thickeners, etc.

### Prepolymers Suitable for this Invention

Prepolymers suitable for use in the present invention are isocyanate-capped polyether prepolymers with an NCO functionality of greater than 1%. The prepolymers are based on polyether polyols capped with aromatic isocyanates such as, for example, toluene diisocyanate (TDI) or methylene diphenyl isocyanate (MDI), or with aliphatic isocyanates, for example, isopherone diisocyanate (IPDI) or hydrogenated methylene diphenyl isocyanate (HMDI). The polyether polyols are hydrophilic polyoxyalkylenes with a minimum of 40 mole % ethylene oxide. Crosslinking sites are developed, when necessary, during the prepolymer formation by:

the addition of water to the prepolymer polyols to form urea and subsequently biuret linkages in the prepolymer.

the formation of allophate linkages by prolonged heating at elevated temperatures.

the branching of prepolymers by the addition of triols or tetrols. (for example, trimethylolpropane, glycerol, or pentaerythritol).

the formation of branches by the use of selective catalysts.

Isocyanate-capped polyether prepolymers that have been found to be suitable for use in the practice of the present invention include prepolymers sold by the following companies:

Lendell Manufacturing, St Charles, Mich.,

Rynel Ltd. Inc Boothbay Me.,

Dow Chemical, Midland Mich.

Mace, Dudley, Mass.

The following table (taken from product literature, lists their products and the NCO specification.

| | % NCO |
|---|---|
| Rynel Polymer Type | |
| B-1 | 7.4-8.32 |
| A-62 | 10.0-11.4 |
| Trepol | 5.2-6.4 |
| Mace Adhesives & Coatings | |
| Bipol | 5.5-6.5 |
| Dow Chemical | |
| Hypol 2000 | 6.3-7.2 |
| Hypol 2002 | 6.3-7.2 |
| Hypol 3000 | 9.5-10.3 |
| Hypol 5000 | 9.5-11.13 |
| Lendell Manufacturing | |
| Prepol | 8.0-8.5 |

It is preferred that the prepolymer have an isocyanate concentration of between 1 and 14% NCO. Most preferred is a concentration between 3 and 11.5% NCO.

### Active Ingredients Suitable for this Invention

It is known that hydrophilic foams will absorb any polar solvent. It is preferred that the active ingredient be solubilized before addition to the foam Solvents such as water, glycerin, alcohols and ethers are useful. Active ingredients that are dispersible in these solvents are also within the scope of this invention.

### EXAMPLE I

A hydrophilic polyurethane foam was obtained from a manufacturer (Rynel, Ltd. Boothbay, Me., Part No. 42395-040). This foam is a so-called medical grade and is produced using a 0.05% solution of Pluronic L62.

The foam was cut into 1.5″ circles and to each circle, 0.75 grams of a specially prepared emulsion was applied. The emulsion was composed of glycerine (USP) and several components that were reported to be beneficial to the skin. The hydrophilic foam absorbed the emulsion or otherwise took it into its matrix.

The foam circles were packaged individually in foil envelopes and were used in a study to determine the efficacy of the formulation and the acceptability of the delivery system.

Twenty-two individuals, aged 30 to 45, were asked to apply the emulsion to the skin by rubbing the foam sponge on their faces around the mouth and eyes. Each week they were asked to fill out a questionnaire that inquired as to their opinion of the product (emulsion formulation and the sponge applicator).

After 42 days they were asked specifically if the sponge was an effective delivery system. They were to answer by the following number system.

1=Strongly Disagree

2=Mildly Disagree

3=Neither Agree or Disagree

4=Mildly Agree

5=Strongly Agree

The average of the participants was 4.2 (SD=1) indicating agreement that the sponge was an effective delivery system.

An Example for Use as an Eye Treatment:

A formulation for the cosmetic treatment of wrinkles around the eye was composed of a number of ingredients. Each of the ingredients had been tested individually for efficacy in the treatment of skin difficulties. The formulation was dissolved in a quantity of water. A sheet of hydrophilic polyurethane was immersed in the water solution and allowed to soak up the liquid. The foam was then removed and squeezed to remove the excess liquid. The foam was then dried in a conventional tumble drier. After slitting the foam into 1.25″×3″ strips, it was used to make swabs by gluing the foam to the end of a rigid plastic stick.

To test the ability of the device to deliver the ingredients in the formulation, the swabs were wetted with water. After about 2 minutes, the swabs were drawn across a quartz curvette. The curvette was placed in an Ultraviolet Spectrophotometer and the absorption of UV energy measured as a function of wavelength. The spectra of the material deposited on the curvette was identical to a standard made of the formulation indicating that the formulation treated swab was capable of delivering the unadulterated formulation.

An Example for Use in the Treatment of Cellulite:

A formulation for the cosmetic treatment of cellulite on the hips and thigh was composed of a number of ingredients. Each of the ingredients had been tested individually for efficacy in the treatment of skin difficulties. The formulation was dissolved in a quantity of water. A sheet of a hydrophilic polyurethane derivative known as CoFoam® Hydrophilic Polyurethane Composite was supplied by Hydrophilix Corporation, Saco Maine was immersed in the water solution and allowed to soak up the liquid. CoFoam® Hydrophilic Polyurethane Composite is a construction of hydrophilic polyurethane grafted onto a scaffold of substantially open-cell hydrophobic polyurethane. The foam was then removed and squeezed to remove the excess liquid. The foam was then cut into 4″ by 4″ squares and dried in a conventional tumble drier. The squares were tested for the ability of the pad to deliver the unadulterated formulation by the same method as with the eye formulation.

That test confirmed the correct delivery of ingredients.

The product was also used in a trial in which women were asked to wet the squares and rub it on their hips and thighs where the appearance of cellulite was evident. The procedure was conducted daily for 14 days and the participants were asked to comment on any improvements. All the women indicated that improvements were evident.

While certain representative embodiments of the invention have been described herein for the purposes of illustration, it will be apparent to those skilled in the art that modification therein may be made without departure from the spirit and scope of the invention.

What is claimed is:

1. A foam pad delivery pad consisting essentially of:
a hydrophilic polyurethane foam;
at least one active ingredient substantially without any inactive ingredients wherein said foam stabilizes said at least one active ingredient such said at least one active ingredient can be delivered to a portion of the skin of a user for the purpose of affecting a positive effect on the appearance of the skin of the user.

2. A method of manufacturing a foam deliver pad comprising the steps of:
fabricating the foam pad using essentially hydrophilic urethane foam;
washing the foam pad to remove any residual surfactants that were used to make the hydrophilic urethane foam; and
imbibing at least one active ingredient substantially without any inactive ingredients in said foam.

3. The method of claim 2 wherein said imbibing step occurs by having said at least one inactive ingredient soaked in the foam from a solution.

4. The method of claim 2 wherein said imbibing step occurs by having said at least one inactive ingredient soaked in the foam from a dispersion.
